(19) United States
(12) Patent Application Publication
(10) Pub. No.: US 2026/0198757 A1
(43) Pub. Date: Jul. 16, 2026

(54) IMAGE-PROCESSING DEVICE, IMAGE-PROCESSING METHOD, AND RECORDING MEDIUM

(71) Applicant: OLYMPUS MEDICAL SYSTEMS CORP., Tokyo (JP)

(72) Inventors: Taketo TSUKIOKA, Hachioji-shi (JP); Kai TANAKA, Nishitokyo-shi (JP)

(73) Assignee: OLYMPUS MEDICAL SYSTEMS CORP., Tokyo (JP)

(21) Appl. No.: 19/563,143

(22) Filed: Mar. 11, 2026

Related U.S. Application Data
(63) Continuation of application No. PCT/JP2023/035088, filed on Sep. 27, 2023.

(51) Int. Cl. A61B 1/00

(52) U.S. Cl. CPC A61B 1/000096; A61B 1/000095; A61B 1/00013; A61B 1/0004; A61B 1/00045; A61B 1/00055

(57) ABSTRACT

An image-processing device includes a processor. The processor determines a state of an endoscope. When an imaging field of view of an image sensor of the endoscope is divided into a first region including the center of the imaging field of view and a second region other than the first region and the endoscope is inserted into an examination target, the processor executes image processing of setting a visibility of an image of the second region to be lower than a visibility of an image of the first region. When the endoscope is inserted into the examination target, the processor executes image processing of setting a detection performance for an object in the image of the second region to be higher than or equal to a detection performance for an object in the image of the first region.

### BACKGROUND OF THE INVENTION

### FIELD OF THE INVENTION

The present invention relates to an image-processing device, an image-processing method, and a recording medium.

This application is a continuation application based on PCT International Patent Application No. PCT/JP2023/035088, filed September 27, 2023, the content of which is incorporated herein by reference.

### DESCRIPTION OF RELATED ART

An endoscope is an examination device in which a very small camera is attached to the distal end and is inserted into an examination target such as a lumen or a narrow opening. A user observes an object by observing an image generated by an imaging unit at the distal end of the endoscope. The user moves the endoscope in an insertion direction in which the endoscope moves inward in an examination target or in an extraction direction in which the endoscope moves outward in the examination target. An image output from the imaging unit at the time of insertion of the endoscope is used to assist with insertion of the endoscope. Detailed checkup of the inside of an examination target in an examination of a large intestine or the like may be performed at the time of extraction of the endoscope. This is for causing the endoscope to reach the deepest position in the examination target and preventing any overlooking overall.

The role of an image at the time of insertion of the endoscope may be different from the role of an image at the time of an examination or at the time of extraction of the endoscope. When the endoscope is inserted into an examination target, an image is effectively used to assist with insertion of the endoscope. At this time, the user views a central region of an image generated by the imaging unit and checks that the endoscope moves along the hole of a lumen. The user causes the endoscope to advance in the direction checked in the image. In this situation, when an imaging field of view is wider than necessary, the amount of information of a peripheral region of the image is large. Accordingly, the user viewing the center of the image may feel stressed. Here, a lesioned part or the like may appear in the peripheral region of the image, and thus there is a likelihood that the user viewing the center of the image may overlook the lesioned part or the like.

Japanese Unexamined Patent Application, First Publication No. 2021-051470 discloses a technique of preventing overlooking of an object by allowing a plurality of cameras installed in a vehicle to cooperate with each other. In this technique, for example, a first object-tracking unit tracks an object in an image, and a second object-tracking unit tracks the object when tracking using the first object-tracking unit becomes impossible or inappropriate, whereby a range of the image is switched according to the purpose.

### SUMMARY OF THE INVENTION

According to a first aspect of the present invention, an image-processing device includes a processor. The processor determines whether a state of an endoscope is either a first state in which the endoscope is advancing inward in an examination target or a second state other than the first state. When an imaging field of view of an image sensor of the endoscope is divided into a first region including a center of the imaging field of view and a second region other than the first region and the state of the endoscope is the first state, the processor executes image processing of setting a visibility of an image of the second region to be lower than a visibility of an image of the first region to generate an image for display. The processor executes image processing of setting a detection performance for an object in the image of the second region to be higher than or equal to a detection performance for an object in the image of the first region to generate an image for object detection when the state of the endoscope is the first state.

According to a second aspect of the present invention, in the first aspect, the processor may not execute the image processing of setting the visibility of the image of the second region to be lower than the visibility of the image of the first region when the state of the endoscope is the second state.

According to a third aspect of the present invention, in the first aspect, the processor may detect an object from the image of the second region in the image for object detection when the state of the endoscope is the first state. The processor may display a detection result indicating that the object has been detected along with the image of the first region on which the image processing has been executed on a display when the state of the endoscope is the first state.

According to a fourth aspect of the present invention, in the third aspect, the processor may display position information indicating a position of the object in the image of the second region as the detection result on the display.

According to a fifth aspect of the present invention, in the first aspect, the processor may detect an object from the image of the second region in the image for object detection and store first position information indicating a position of the object in the image of the second region and second position information indicating a position of the object in the examination target on a recording medium when the state of the endoscope is the first state. The processor may display the first position information along with an image generated by the image sensor on a display when the state of the endoscope is the second state and a position of the endoscope matches the position indicated by the second position information.

According to a sixth aspect of the present invention, in the first aspect, the processor may delete the image of the second region when the state of the endoscope is the first state.

According to a seventh aspect of the present invention, in the sixth aspect, the processor may detect an object from the image of the second region in the image for object detection when the state of the endoscope is the first state. The processor may delete the image of the second region when the object has not been detected. The processor may not delete the image of the second region when the object has been detected.

According to an eighth aspect of the present invention, in the sixth aspect, the processor may detect an object from the image of the second region in the image for object detection when the state of the endoscope is the first state. The first region in a plurality of images consecutively generated by the image sensor may be gradually enlarged and the second region in the plurality of images may be gradually reduced when the object in the plurality of images approaches the center of the imaging field of view.

According to a ninth aspect of the present invention, in the first aspect, the processor may execute a thinning-out process on the image of the second region when the state of the endoscope is the first state.

According to a tenth aspect of the present invention, in the first aspect, the processor may correct an image generated by the image sensor based on optical characteristics of the endoscope.

According to an eleventh aspect of the present invention, an image-processing device includes a processor. When an imaging field of view of an image sensor of an endoscope is divided into a first region including a center of an imaging field of view and a second region other than the first region, the processor executes first image processing on an image of the first region generated by the image sensor. The processor executes second image processing on an image of the second region generated by the image sensor. The processor detects an object in the image on which the second image processing has been executed. The processor displays a detection result of the object along with the image of the first region on which the first image processing has been executed on a display.

According to a twelfth aspect of the present invention, in the eleventh aspect, a boundary between the first region and the second region may be switchable between that in a first state in which the endoscope is inserted into an examination target and is caused to advance inwardly in the examination target and that in a second state in which the endoscope is extracted from the examination target.

According to a thirteenth aspect of the present invention, an image-processing device includes a processor. When an imaging field of view of an image sensor of an endoscope is divided into a first region including a center of the imaging field of view and a second region other than the first region, the processor acquires an inference model, which is obtained through machine learning using a first learning image and a first annotation as first training data and using a second learning image and a second annotation as second training data, from a recording medium. First image processing has been executed on the first learning image. The first annotation indicates an object in the first region of the first learning image. Second image processing has been executed on the second learning image. The second annotation indicates an object in the second region of the second learning image. The processor detects an object in an image generated by the image sensor using the inference model.

According to a fourteenth aspect of the present invention, an image-processing device includes a processor. When an imaging field of view of an image sensor of an endoscope is divided into a first region including a center of the imaging field of view and a second region other than the first region, the processor acquires a first inference model, which is obtained through first machine learning using a learning image generated by an image sensor of an endoscope and a first annotation indicating an object in the first region of the learning image as first training data, from a recording medium. The processor acquires a second inference model, which is obtained through second machine learning using the learning image and a second annotation indicating an object in the second region of the learning image as second training data, from the recording medium. The processor detects an object in an image generated by the image sensor using the first inference model and the second inference model.

According to a fifteenth aspect of the present invention, an image-processing method includes: determining whether a state of an endoscope is either a first state in which the endoscope is advancing inward in an examination target or a second state other than the first state; when an imaging field of view of an image sensor of the endoscope is divided into a first region including a center of the imaging field of view and a second region other than the first region and the state of the endoscope is the first state, executing image processing of setting a visibility of an image of the second region to be lower than a visibility of an image of the first region to generate an image for display; and executing image processing of setting a detection performance for an object in the image of the second region to be higher than or equal to a detection performance for an object in the image of the first region to generate an image for object detection when the state of the endoscope is the first state.

According to a sixteenth aspect of the present invention, an image-processing method includes: when an imaging field of view of an image sensor of an endoscope is divided into a first region including a center of the imaging field of view and a second region other than the first region, executing first image processing on an image of the first region generated by the image sensor; executing second image processing on an image of the second region generated by the image sensor; detecting an object in the image on which the second image processing has been executed; and displaying a detection result of the object along with the image of the first region on which the first image processing has been executed on a display.

According to a seventeenth aspect of the present invention, an image-processing method includes: when an imaging field of view of an image sensor of an endoscope is divided into a central region including a center of the imaging field of view and a peripheral region other than the central region, executing image processing for display on an image of the central region generated by the image sensor; detecting an object in an image of the peripheral region generated by the image sensor; and displaying a detection result of the object on a display on which the image of the central region is displayed.

According to an eighteenth aspect of the present invention, in the seventeenth aspect, the image of the central region may have a rectangular shape. The detection result of the object may be an icon including at least one of information indicating presence of the object, information indicating a type of the object, and information indicating a direction directed from a center of the rectangular shape to a side closest to a position at which the object has been detected out of four sides of the rectangular shape.

According to a nineteenth aspect of the present invention, a non-transitory computer-readable recording medium stores a program causing a computer to execute: determining whether a state of an endoscope is either a first state in which the endoscope is advancing inward in an examination target or a second state other than the first state; when an imaging field of view of an image sensor of the endoscope is divided into a first region including a center of the imaging field of view and a second region other than the first region and the state of the endoscope is the first state, executing image processing of setting a visibility of an image of the second region to be lower than a visibility of an image of the first region to generate an image for display; and executing image processing of setting a detection performance for an object in the image of the second region to be higher than or equal to a detection performance for an object in the image of the first region to generate an image for object detection when the state of the endoscope is the first state.

According to a twentieth aspect of the present invention, a non-transitory computer-readable recording medium stores a program causing a computer to execute: when an imaging field of view of an image sensor of an endoscope is divided into a first region including a center of the imaging field of view and a second region other than the first region, executing first image processing on an image of the first region generated by the image sensor; executing second image processing on an image of the second region generated by the image sensor; detecting an object in the image on which the second image processing has been executed; and displaying a detection result of the object along with the image of the first region on which the first image processing has been executed on a display.

### BRIEF DESCRIPTION OF THE DRAWINGS

FIG. 1 is a block diagram showing an example of the configuration of an endoscope system according to an embodiment of the present invention.

FIG. 2 is a block diagram showing an example of the configuration of an imaging unit included in the endoscope system according to the embodiment of the present invention.

FIG. 3 is a diagram showing an example of the configuration of the imaging unit included in the endoscope system according to the embodiment of the present invention.

FIG. 4 is a diagram showing the principle of image correction in the embodiment of the present invention.

FIG. 5A is a diagram showing an example of an insertion state in the embodiment of the present invention.

FIG. 5B is a diagram showing an example of an extraction state in the embodiment of the present invention.

FIG. 6A is a diagram showing an example of an examination state in the embodiment of the present invention.

FIG. 6B is a diagram showing an example of the examination state in the embodiment of the present invention.

FIG. 7 is a flowchart showing an example of a procedure of a process executed by an image-processing device according to the embodiment of the present invention.

FIG. 8 is a flowchart showing an example of a procedure of a process executed by the image-processing device according to the embodiment of the present invention.

FIG. 9A is a diagram showing an example of an image in the embodiment of the present invention.

FIG. 9B is a diagram showing an example of an image in the embodiment of the present invention.

FIG. 9C is a diagram showing an example of an image in the embodiment of the present invention.

FIG. 10A is a diagram showing an example of an image in the embodiment of the present invention.

FIG. 10B is a diagram showing an example of an image in the embodiment of the present invention.

FIG. 11A is a diagram showing an example of an image in the embodiment of the present invention.

FIG. 11B is a diagram showing an example of an image in the embodiment of the present invention.

FIG. 12 is a diagram showing an example of an image in the embodiment of the present invention.

FIG. 13 is a block diagram showing an example of the configuration of an inference model generation unit included in the endoscope system according to the embodiment of the present invention.

### DETAILED DESCRIPTION OF THE INVENTION

Hereinafter, embodiments of the present invention will be described with reference to the drawings. In the following description, an example of an endoscope system including an image-processing device will be described. “A plurality of XX” in the following description means two or more XX.

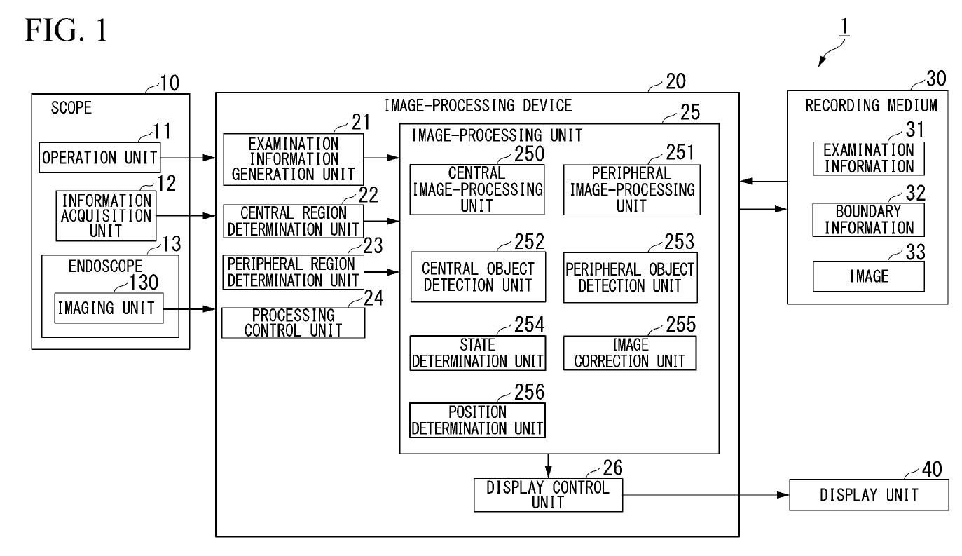

FIG. 1 shows an example of the configuration of an endoscope system 1 according to an embodiment of the present invention. The endoscope system 1 shown in FIG. 1 includes a scope 10, an image-processing device 20, a recording medium 30, and a display unit 40.

The scope 10 includes an operation unit 11, an information acquisition unit 12, and an endoscope 13. The endoscope 13 has a thin and long tubular shape, and a distal end portion of the endoscope 13 is bendable. The operation unit 11 includes a knob (lever) that is operated to bend the distal end of the endoscope 13. The information acquisition unit 12 outputs a signal to the image-processing device 20 according to a result of operation of the operation unit 11.

The lever is provided to correspond to an X direction and a Y direction in order to bend the distal end of the endoscope 13 in a direction corresponding to the X direction or the Y direction in an image generated by an imaging unit 130 included in the endoscope 13. In general, the X-direction lever can fall in the X direction (laterally), and the Y-direction lever can fall in the Y direction (vertically). It is difficult to determine which of the X-direction lever and the Y-direction lever is to fall, in what direction the lever is to fall, and how the lever is to fall. A user performs an insertion operation through this operation, and it is important to assist with this operation.

The endoscope 13 is inserted into an examination target. The examination target is an internal organ of an examinee. For example, the internal organ is an intestine such as the large intestine or the stomach. The endoscope 13 includes the imaging unit 130. The imaging unit 130 is disposed at the distal end of the endoscope 13. The imaging unit 130 includes an image sensor and generates a plurality of images at a plurality of positions in the intestine of the examinee. The imaging unit 130 outputs the plurality of images to the image-processing device 20. A user such as a doctor inserts the endoscope 13 into the intestine of the examinee and causes the endoscope 13 to advance to a predetermined position. Thereafter, the user performs an examination while slowly bending or drawing the endoscope 13.

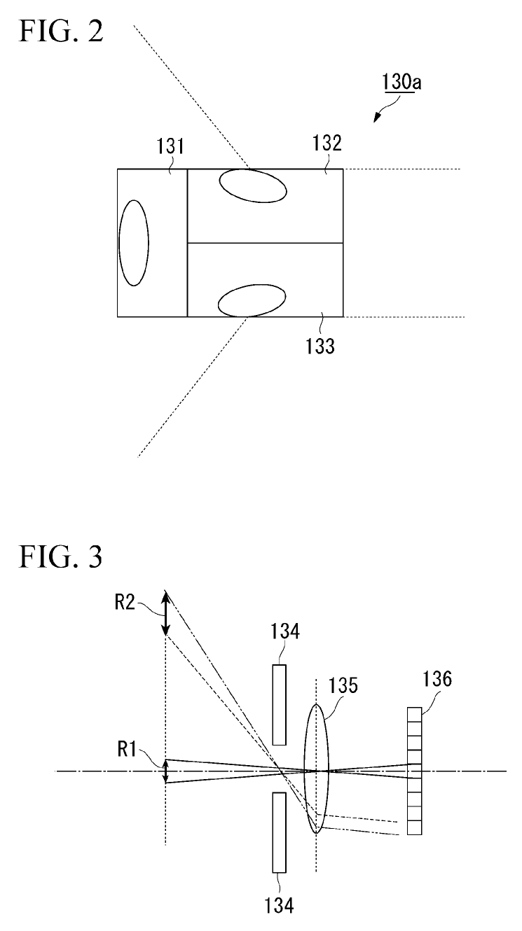

The imaging unit 130 may be replaced with an imaging unit 130a shown in FIG. 2. The imaging unit 130a includes a forward-view imaging unit 131, a rearward-view imaging unit 132, and a rearward-view imaging unit 133. The forward-view imaging unit 131 generates an image of an object in front of the endoscope 13. The image generated by the forward-view imaging unit 131 corresponds to an image of a central region that will be described later. The rearward-view imaging unit 132 and the rearward-view imaging unit 133 generate an image of an object behind the endoscope 13. Images generated by the rearward-view imaging unit 132 and the rearward-view imaging unit 133 correspond to images of a peripheral region that will be described later.

When an endoscope having a rearward imaging field of view in addition to a forward imaging field of view is used in this way and the imaging field of view is wider than necessary as described above, the amount of information on the peripheral region of the image increases, and a user gazing at the center of the image is likely to feel stressed.

The forward-view imaging unit 131, the rearward-view imaging unit 132, and the rearward-view imaging unit 133 may generate images at the same time. The endoscope system 1 may switch between a mode in which the forward-view imaging unit 131 generates an image and a mode in which the rearward-view imaging unit 132 and the rearward-view imaging unit 133 generate an image.

The image-processing device 20 includes an examination information generation unit 21, a central region determination unit 22, a peripheral region determination unit 23, a processing control unit 24, an image-processing unit 25, and a display control unit 26.

The examination information generation unit 21 generates examination information indicating examination conditions based on information input according to an operation of an operation unit not shown in FIG. 1. For example, the examination information includes sex of an examinee, age of the examinee, and an examination target. The examination target indicates the type of an intestine (such as a large intestine or the stomach). The examination information generated by the examination information generation unit 21 is recorded as examination information 31 on a recording medium 30.

The central region determination unit 22 determines a central region in an image based on boundary information 32 recorded on the recording medium 30. The boundary information 32 indicates the position of a boundary between an image of a central region and an image of a peripheral region generated by the imaging unit 130 when the imaging field of view of the imaging unit 130 is divided into the central region (a first region) including the center of the imaging field of view and the peripheral region (a second region) other than the central region. The peripheral region is a region outside the central region and surrounds the central region. For example, the entire image and the image of the central region generated by the imaging unit 130 have a rectangular shape. The boundary between the central region and the peripheral region has a rectangular shape. The central region determination unit 22 outputs information indicating the position of the central region to the image-processing unit 25.

The peripheral region determination unit 23 determines a peripheral region in an image based on the boundary information 32 recorded on the recording medium 30. The peripheral region determination unit 23 outputs information indicating the position of the peripheral region to the image-processing unit 25.

The boundary between the central region and the peripheral region in an insertion state (a first state) may be the same as the boundary between the central region and the peripheral region in an extraction state (a second state). The insertion state is a state in which the endoscope 13 is inserted into an examination target and the endoscope 13 is advancing inwardly in the examination target. The extraction state is a state in which the endoscope 13 is being extracted from the examination target. As will be described later, the boundary between the central region and the peripheral region in the insertion state may be different from the boundary between the central region and the peripheral region in the extraction state. That is, the boundary between the central region and the peripheral region may switch between the insertion state and the extraction state.

Although it also depends on design, when an angle of view is greater than or equal to 60°, an influence of aberration is conspicuous due to general characteristics of a lens. Accordingly, the boundary between the central region and the peripheral region may be set to a position corresponding to an angle of view of 60°.

The endoscope system 1 switches control for each region in an image corresponding to a region of the imaging field of view of the imaging unit 130 and executes processing according to the role of the image required in the insertion state and the other states.

The processing control unit 24 controls the examination information generation unit 21, the central region determination unit 22, the peripheral region determination unit 23, the image-processing unit 25, and the display control unit 26. In addition, the processing control unit 24 records an image output from the imaging unit 130 as an image 33 on the recording medium 30.

The image-processing unit 25 processes an image generated by the imaging unit 130. The image-processing unit 25 includes a central image-processing unit 250, a peripheral image-processing unit 251, a central object detection unit 252, a peripheral object detection unit 253, a state determination unit 254, an image correction unit 255, and a position determination unit 256.

The central image-processing unit 250 (a first image-processing unit) executes image processing (central image processing) on the image of the central region determined by the central region determination unit 22. The peripheral image-processing unit 251 (a second image-processing unit) executes image processing (peripheral image processing) on the image of the peripheral region determined by the peripheral region determination unit 23. The peripheral image processing (second image processing) may be the same as the central image processing (first image processing), or the peripheral image processing may be different from the central image processing. An imaging mode set in the imaging unit 130 may switch between the insertion state and the extraction state, and the central image processing and the peripheral image processing may be set according to the imaging mode.

The following switching is conceivable as switching of the imaging mode between the insertion state and the extraction state. In addition, in order to check an insertion direction in front of the imaging unit 130 and a lumen direction in the insertion state and to avoid collision, the image of the central region is important. When the quality and visibility of the image are poor, correct and safe insertion is difficult.

Based on an idea that information of the image of the central region in the insertion state is important, an idea that reducing the amount of information of the image of the peripheral region relatively is allowable can also be derived. This idea may be particularly described, but this idea is a result of emphasis of features of the invention and is not necessarily applied to any situation.

On the other hand, in the extraction state, the distal end of the endoscope 13 is less likely to collide with a lumen wall. It is rather important to determine the type of an object moving in a direction opposite to the moving direction of the endoscope 13 in the extraction state, that is, from the periphery of the image to the center of the image. When the extraction speed is high, an object appearing in the periphery of the image instantaneously goes away from the distal end of the endoscope 13 and becomes smaller than a visible size. Accordingly, a user may miss a significant region. In order to utilize the feature that an object appears large in the image of the peripheral region, there is need for ingenuity for improving the visibility and quality of the image of the peripheral region in the extraction state.

When the user visually inserts the endoscope 13 into an examination target, image processing that places a priority on a visibility and includes correct control of exposure, focusing, color reproducibility, and the like is significant. On the other hand, when a computer executes automated insertion or insertion assistance based on an image, image processing of increasing the amount of information acquired from the image or the like is also significant. Other than image processing, there are also factors such as driving of a lens for focusing or illumination control. In addition, unlike the central region, improvement in image quality in the peripheral region is not easy due to constraints in design of an optical system and an imaging element or the like, and thus an idea for improving image quality is also significant.

For example, when the state of the endoscope 13 is the insertion state, the peripheral image-processing unit 251 (the first image-processing unit) executes image processing of setting a visibility of the image of the peripheral region in the image generated by the imaging unit 130 to be lower than the visibility of the central region in the image generated by the imaging unit 130 and generates an image for display. For example, the peripheral image-processing unit 251 deletes the image of the peripheral region from the image generated by the imaging unit 130. Alternatively, the peripheral image-processing unit 251 executes a thinning-out process on the image generated by the imaging unit 130 and thins out pixels of the image of the peripheral region. Accordingly, the number of pixels in the peripheral region also decreases.

The central image-processing unit 250 may execute image processing of increasing the visibility of the image of the central region. This processing can also be referred to as image processing in consideration of a visibility such that a user can easily check the image of the central region when the image is displayed on the display unit 40. That is, it is assumed that an image with natural brightness, color expression, and gradation expression and with an appropriate dynamic range and contrast is generated through this processing. The visibility may become worse due to liquids, bubbles, and the like in the biological body, and the central image-processing unit 250 may execute correction of removing them. The central image-processing unit 250 may execute correction according to performance such as an aspect ratio or the number of pixels of a display, brightness conditions in the environment, or the like. In addition, an adaptive processing technique of recognizing features for each region of an image and executing appropriate image correction based thereon may be used together. It is also important to display a large and clear image on a display screen such that a user located at a far position can check the image.

When the state of the endoscope 13 is the insertion state, the peripheral image-processing unit 251 (the second image-processing unit) executes image processing of setting the detection performance for an object in the image of the peripheral region to be greater than or equal to the detection performance for an object in the image of the central region and generates an image for object detection. For example, the object is a lesioned part. For example, the detection performance for an object is a minimum size of an object that is detectable or detection speed of the object.

For example, the central image-processing unit 250 executes processing of improving the image quality on the image of the central region, and the peripheral image-processing unit 251 executes processing of improving the image quality on the image of the peripheral region. The processing of improving the image quality includes noise reduction, edge emphasis, color adjustment, or high dynamic range (HDR) processing.

The intensity of the processing executed on the image of the peripheral region is greater than or equal to the intensity of the processing executed on the image of the central region. Accordingly, the effect of improvement in image quality in the image of the peripheral region is higher than or equal to the effect of improvement in image quality in the image of the central region. That is, the detection performance for an object in the image of the peripheral region is greater than or equal to the detection performance for an object in the image of the central region.

The processing of generating an image for object detection is image processing based on the assumption that a user does not have to check the image of the peripheral region when the image is displayed on the display unit 40. Since it is assumed that the peripheral object detection unit 253 detects an object in the image of the peripheral region, this image processing is possible. Accordingly, in the processing of generating an image for display, image processing of setting the visibility of the peripheral region to be lower than the visibility of the central region can be executed. As such image processing, image processing of decreasing brightness, decreasing a color chroma, and moderating a dynamic range and a contrast may be used. The central image-processing unit 250 and the peripheral image-processing unit 251 may execute at least one of the aforementioned image processing or execute other means to generate an image for display.

The visibility may become worse due to liquids, bubbles, and the like in the biological body, and correction of removing them is important to detect an object. Accordingly, the central image-processing unit 250 and the peripheral image-processing unit 251 may execute the correction in the processing of generating the image for object detection. The central image-processing unit 250 and the peripheral image-processing unit 251 may execute the correction according to performance such as an aspect ratio or the number of pixels of a display, brightness conditions in the environment, or the like. Processing of curbing the visibility may be used as the correction. In addition, an adaptive processing technique of recognizing features for each region of an image and executing appropriate image correction based thereon may be used together. Processing of curbing the visibility may be used as the correction. Since a visibility to the user does not have to be considered, the central image-processing unit 250 and the peripheral image-processing unit 251 may reduce or deform an image.

In the insertion state, a user gazes at the image of the central region. Since the visibility of the image of the peripheral region in the image for display is lower than the visibility of the image of the central region, a user is less likely to feel stressed due to an influence of the image of the peripheral region. In addition, since the detection performance for an object in the image of the peripheral region in the image for object detection is higher than or equal to the detection performance for an object in the image of the central region, the user is less likely to overlook an object in the peripheral region.

For example, when the state of the endoscope 13 is the extraction state, the peripheral image-processing unit 251 does not execute the image processing of setting the visibility of the image of the peripheral region to be lower than the visibility of the image of the central region and executes image processing of setting the visibility of the image of the peripheral region to be equal to the visibility of the image of the central region. In the extraction state, the user observes the image of the central region and the image of the peripheral region. When a lesioned part is detected, the user performs an examination of the lesioned part. The examination of a lesioned part includes diagnosis or treatment of the lesioned part. Since the visibility of the image of the peripheral region is equal to the visibility of the image of the central region, the user is less likely to overlook an object in the peripheral region.

The central object detection unit 252 detects a feature region having a predetermined feature as an object from the image of the central region in the image for object detection. Techniques of detecting a face to adjusti focus and exposure using a general camera may be applied to the processing of detecting the feature region. Pattern matching or an inference model may be used. When an inference model obtained through learning using an image in which an object such as a lesioned part appears and an annotation that is position information of the object as training data is used, an endoscopic image is input to the central object detection unit 252. The central object detection unit 252 outputs information indicating whether an object is present in the image and outputs position information of an object when the object has been detected in the image.

The peripheral object detection unit 253 detects a feature region having a predetermined feature as an object from the image of the peripheral region in the image for object detection. Techniques of detecting a face to adjust focus and exposure using a general camera may be applied to the processing of detecting the feature region as described above. When an inference model receiving an input of an endoscopic image, outputting information indicating whether an object is present in the image, and outputting position information of an object when the object has been detected in the image is used, an effect that AI technology instead of a medical employee monitors an image can be achieved. When learning using the type, the size, and other features of an object in addition to the position information as an annotation is executed, such information can also be inferred.

When the position of an object is known, a direction directed from the center of a rectangular image to a side closest to the position at which the object has been detected out of four sides of the rectangular shape can be calculated. For example, when the position in an image is represented in a coordinate system with the center of the image as an origin, the horizontal direction of the image can be defined as an X direction and the vertical direction of the image can be defined as a Y direction. That is, when the coordinate of an object in the X direction exceeds the range of the central region in the X direction, the object is located outside the central region and on the left or right side of the central region. When the coordinate of the object in the Y direction exceeds the range of the central region in the Y direction, the object is located outside the central region and above or below the central region.

The state determination unit 254 determines whether the state of the endoscope 13 is either an insertion state, an extraction state, or an examination state (an observation state). In the insertion state, an image feature of a lumen wall moves radially to the periphery in a plurality of images acquired in a time series. While the endoscope 13 is being inserted, a user may direct the distal end of the endoscope 13 to a side other than the insertion direction, be confused, or cause the distal end to stop in order to check the insertion direction. When the radial movement can be detected as a feature of a motion vector even in such a situation, the state determination unit 254 may determine that the state of the endoscope 13 is the insertion state.

When an acceleration sensor is provided at the distal end of the endoscope 13, the state determination unit 254 may determine an acceleration direction based on a signal output from the acceleration sensor and determine that the state of the endoscope 13 is the insertion state. When a predetermined sequence is scheduled, the state determination unit 254 may determine that the state of the endoscope 13 is the insertion state in a scheduled time period. Until a deepest object appears in the image, the state determination unit 254 may determine that the state of the endoscope 13 is the insertion state.

The examination state is a state in which the distal end of the endoscope 13 is directed to a lesioned part and a user is examining the lesioned part. In the examination state, since an image feature indicating that the distal end of the endoscope 13 approaches a lesioned part is obtained, the state determination unit 254 can determine the examination state based on the image feature. In this case, an image in which the object is enlarged and moving toward the center of the image is obtained, and then a plurality of similar images are consecutively acquired for observation. Accordingly, the state determination unit 254 can determine the examination state based on such features.

The method of determining the extraction state of the endoscope 13 is the same as the method of determining the state of the endoscope 13. An image feature of a lumen wall moves radially to the center in a plurality of images acquired in a time series. While the endoscope 13 is being inserted, a user may direct the distal end of the endoscope 13 to a side other than the extraction direction or cause the distal end, be confused, or cause the distal end to stop in order to check the insertion direction. When the radial movement can be detected as a feature of a motion vector even in such a situation, the state determination unit 254 may determine that the state of the endoscope 13 is the extraction state.

When an acceleration sensor or the like is provided at the distal end of the endoscope 13, the state determination unit 254 may determine an acceleration direction based on a signal output from the acceleration sensor and determine that the state of the endoscope 13 is the extraction state. When a predetermined sequence is scheduled, the state determination unit 254 may determine that the state of the endoscope 13 is the extraction state in a scheduled time period. Until the endoscope 13 is extracted from the examination target after a deepest object has appeared in the image, the state determination unit 254 may determine that the state of the endoscope 13 is the extraction state.

Even in the examination state while the endoscope 13 is being extracted, since an image feature indicating that the distal end of the endoscope 13 approaches a lesioned part is obtained, the state determination unit 254 can determine the examination state based on the image feature. In this case, an image in which the object is enlarged and moving toward the center of the image is obtained, and then a plurality of similar images are consecutively acquired for observation. Accordingly, the state determination unit 254 can determine the examination state based on such features.

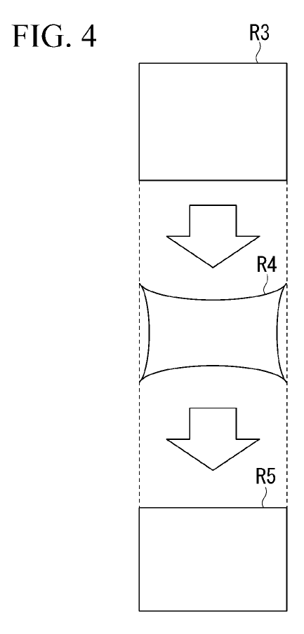

The image correction unit 255 corrects an image generated by the imaging unit 130 based on optical characteristics of the endoscope 13. Details of the process executed by the image correction unit 255 will be described with reference to FIGS. 3 and 4.

FIG. 3 shows an example of the configuration of the imaging unit 130. The imaging unit 130 includes an iris 134, a lens 135, and an imaging element 136. Light from an object passes through the iris 134 and the lens 135 and is incident on the imaging element 136. A plurality of pixels are disposed on an imaging surface of the imaging element 136.

An optical image of a range R1 is formed at a central pixel of the imaging surface of the imaging element 136. An optical image of a range R2 that is wider than the range R1 is formed on a peripheral pixel of the imaging surface of the imaging element 136 due to an influence of aberration of the lens 135. Accordingly, a larger amount of information is acquired in the peripheral pixels than in the central pixels.

FIG. 4 shows the principle of image correction. An optical image of a range R3 in the imaging field of view is formed in a range R4 of the imaging surface of the imaging element 136 due to an influence of aberration of the lens 135. The image correction unit 255 corrects a positional displacement occurring in the image due to the aberration of the lens 135. That is, the image correction unit 255 corrects distortion of the image. Accordingly, the image correction unit 255 acquires the same image as that acquired when the optical image of the range R3 is formed in a range R5 of the imaging surface.

When the imaging unit 130a shown in FIG. 2 is used, each of the forward-view imaging unit 131, the rearward-view imaging unit 132, and the rearward-view imaging unit 133 generates an image. The image correction unit 255 may correct a positional displacement in the image generated by each imaging unit according to optical characteristics of the corresponding imaging unit.

The position determination unit 256 determines a current position of the distal end of the endoscope 13 based on a plurality of images generated by the imaging unit 130 and generates current position information indicating the current position. The position indicates a position in an intestine imaged by the imaging unit 130.

The “current position of the distal end of the endoscope 13” may be a unit of an organ name (a part name) or a subdivided position in the internal organ. For example, the organ name is throat, stomach, or the like in the case of an upper intestine, and the organ name is rectum, transverse colon, or the like in the case of a lower intestine. For example, the subdivided position is a position advanced by about 3 cm in the transverse colon after passing through an S-shaped colon. The “current position of the distal end of the endoscope 13” may be a position in the lumen, a name in the lumen, or a classified position in the lumen. The “current position of the distal end of the endoscope 13” may be a combination of a name in the lumen and a position, a depth, a length, a distance, or the like in a lumen direction in the lumen. A direction perpendicular to a continuation direction (an axial direction) of the lumen may be used according to necessity.

In a bag-shaped organ such as the stomach, the “current position of the distal end of the endoscope 13” may be a region namesor a part name such as the cardia, the gastric fundus, the gastric corpus, the pyloric antrum, and the pylorus from an entrance or may be information indicating an arbitrary position of a corresponding region by coordinates or the like in more detail. This position information may be detected from image features such as tissue or blood vessel specific to the corresponding region. The position may be determined using the change of image features accompanying the change of image frames consecutively acquired at the time of insertion of the endoscope 13 or using a pattern of blood vessels or tissues. The image-processing device 20 may detect a speed (cm/sec) or the like at the time of insertion of the endoscope 13 and convert the speed to the insertion position using time information, but may not detect the speed and use an average value of an insertion speed or the like as a constant when a general doctor inserts the endoscope. The image-processing device 20 may determine a lumen shape using three-dimensional (3D) reconstruction using image information or the like and estimate the current examination position based on the result of determination.

In the following example, the position indicates a predetermined part in a simplified intestine. When a position sensor is provided at the distal end of the endoscope 13, the position determination unit 256 may determine a part that is currently being examined based on information output from the position sensor. For example, there is a method of detecting a part using magnetism or the like. The part may be detected using another medical instrument such as a CT or an MRI. The position determination unit 256 generates part information (current position information) indicating the determined part.

An expression “a position in an internal organ,” may represent a region name in the internal organ such as the cardia, the gastric fundus, the gastric corpus, the pyloric antrum, or the pylorus. The “position in an internal organ” may be used to mean the difference between internal organs in different classifications such as the throat and the stomach.

The display control unit 26 superimposes information to be notified to a user on an image output from the image-processing unit 25 and outputs the image to the display unit 40. Accordingly, the display control unit 26 displays the information along with the image. For example, the display control unit 26 displays a result of detection indicating that an object has been detected along with the image of the central region processed by the central image-processing unit 250 on the display unit 40.

For example, when the state of the endoscope 13 is the insertion state, the peripheral object detection unit 253 detects an object from the image of the peripheral region in the image for object detection. The display control unit 26 displays position information indicating the position of the object in the image of the peripheral region along with the image of the central region processed by the central image-processing unit 250 on the display unit 40. When the image of the peripheral region is deleted by the peripheral image-processing unit 251 or pixels of the image of the peripheral region are thinned out, it is difficult for a user to check an object in the image of the peripheral region. However, the user can ascertain that an object is present at the position indicated by the position information. Accordingly, the user can avoid overlooking of an object.

When the state of the endoscope is the insertion state, the peripheral object detection unit 253 detects an object from the image of the peripheral region in the image for object detection. The peripheral object detection unit 253 stores first position information indicating the position of the object in the image of the peripheral region and second position information indicating the position of the object in the examination target in the recording medium 30. The second position information is the part information generated by the position determination unit 256. The first position information indicates the position of the object in the image generated by the imaging unit 130 at the position indicated by the second position information.

When the state of the endoscope 13 is the extraction state and the position of the endoscope 13 matches the position indicated by the second position information, the display control unit 26 displays the second position information along with the image generated by the imaging unit 130 on the display unit 40. Accordingly, the user is notified of the position of the object detected in the insertion state. The user can ascertain that the object is present at the position indicated by the second position information. As a result, the user can avoid overlooking of an object.

The image-processing device 20 may be constituted by a processor such as a central processing unit (CPU).

A computer may read a program and execute the read program. The program includes instructions for defining operations of the image-processing device 20. That is, the functions of the image-processing device 20 may be realized by software.

The program may be provided, for example, using a “computer-readable recording medium” such as a flash memory. The program may be transmitted from a computer storing the program to the endoscope system 1 via a transmission medium or using carrier waves in the transmission medium. The “transmission medium” for transmitting a program is a medium having a function of transmitting information. The medium having a function of transmitting information includes a network (a communication network) such as the Internet and a communication circuit line (a communication line) such as a telephone line. The program may realize some of the aforementioned functions. The program may be a differential file (a differential program). The aforementioned functions may be realized by combining the differential program with a program stored in advance in the computer.

The processor and the recording medium do not have to be included in one device and may be constituted by linking a plurality of devices having distributed functions. The processor and the recording medium may be provided on cloud (network).

The display unit 40 is a liquid crystal monitor or the like. The display unit 40 sequentially displays images output from the display control unit 26.

The recording medium 30 is a memory. Examination information 31, boundary information 32, and an image 33 are recorded on the recording medium 30. These are read by the image-processing device 20 and are used for processing executed by the image-processing device 20.

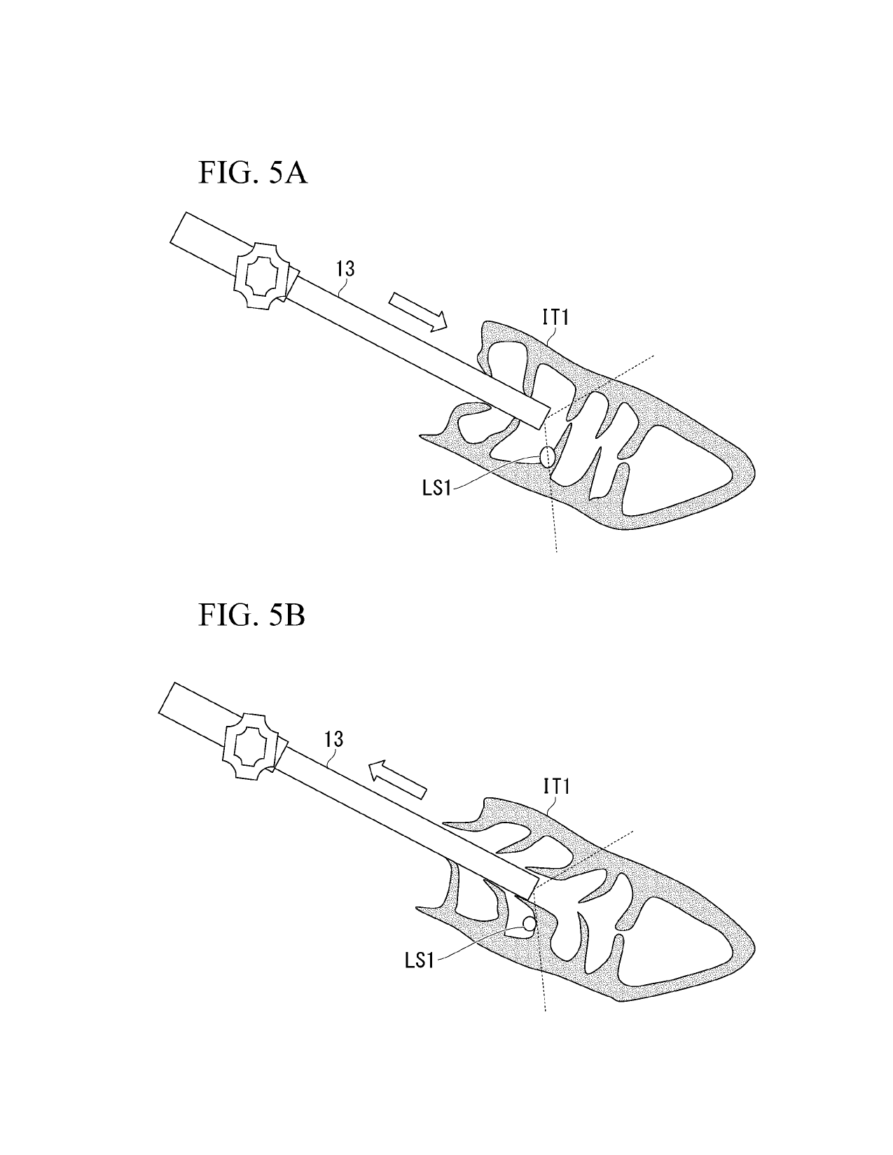

A lesioned part detected in the insertion state may not be detected in the extraction state. FIG. 5A shows an example of the insertion state, and FIG. 5B shows an example of the extraction state. In the insertion state shown in FIG. 5A, the endoscope 13 is inserted into an examination target IT1. As shown in FIG. 5A, a lesioned part LS1 may appear in an image generated in the imaging unit 130 in the insertion state. However, as shown in FIG. 5B, in the extraction state, the lesioned part LS1 may be hidden behind a wall and may not appear in an image generated by the imaging unit 130. In this case, the user is likely to overlook the lesioned part LS1.

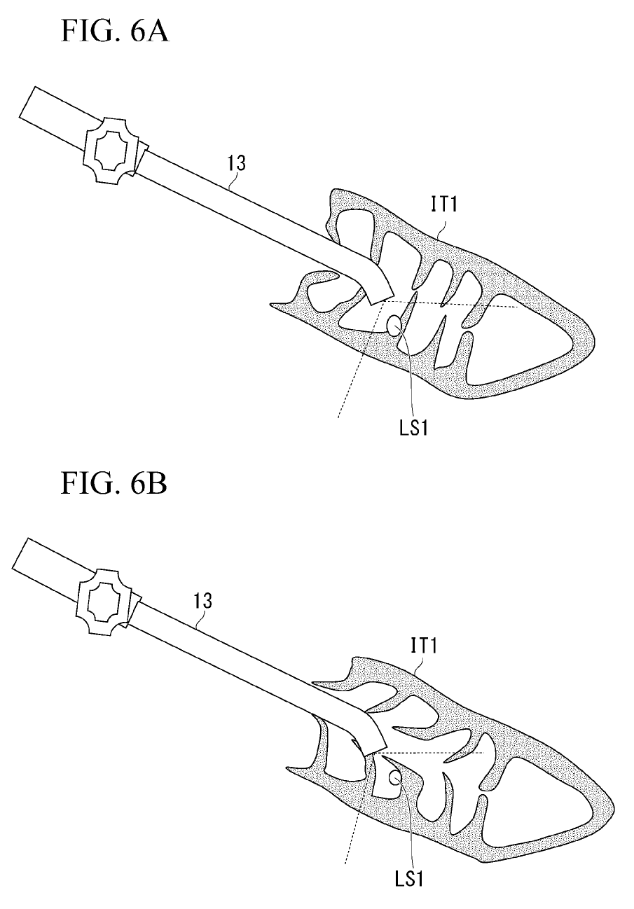

When the state of the endoscope 13 is the insertion state, the display control unit 26 displays position information indicating the position of the lesioned part LS1 in the image of the peripheral region along with the image of the central region processed by the central image-processing unit 250 on the display unit 40. For example, the display unit 40 displays the image of the central region and an arrow indicating the position of the lesioned part LS1. Even when the display unit 40 does not display the image of the peripheral region, the user can ascertain the position of the lesioned part LS1 in the image of the peripheral region. Accordingly, as shown in FIG. 6A, the user can turn the distal end of the endoscope 13 to the lesioned part LS1 and observe the lesioned part LS1. At this time, the state of the endoscope 13 is the examination state.

When the state of the endoscope 13 is the extraction state and the position of the endoscope 13 matches the position of an object in the examination target indicated by the second position information, the display control unit 26 displays first position information indicating the position of the lesioned part LS1 in the image of the peripheral region along with the image generated by the imaging unit 130 on the display unit 40. For example, the display unit 40 displays the image generated by the imaging unit 130 and an arrow indicating the position of the lesioned part LS1. Even when the lesioned part LS1 does not appear in the image as shown in FIG. 5B, the user can ascertain the position of the lesioned part LS1. Accordingly, as shown in FIG. 6B, the user can turn the distal end of the endoscope 13 to the lesioned part LS1 and observe the lesioned part LS1 in a gap between walls. At this time, the state of the endoscope 13 is the examination state.

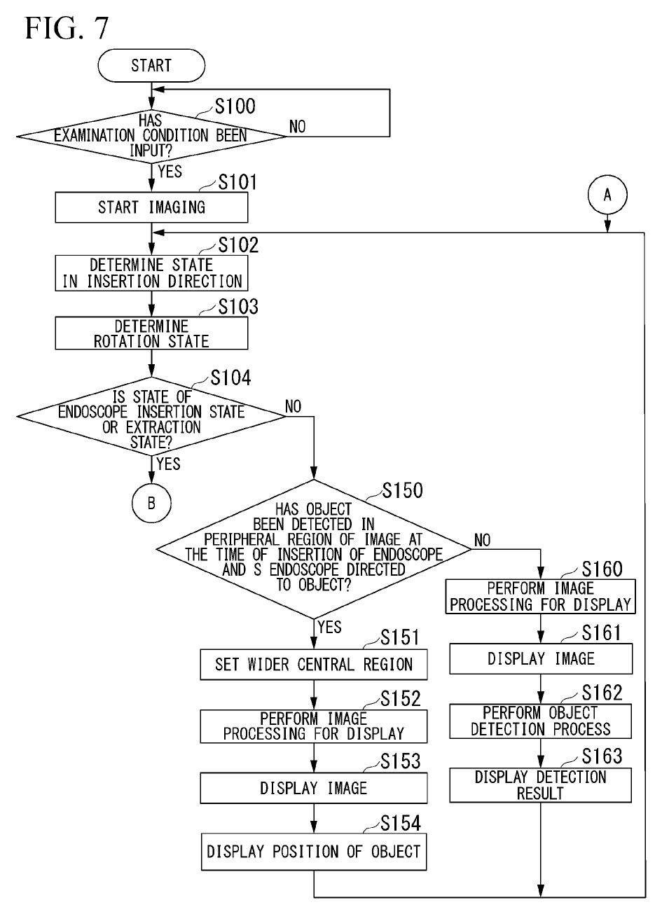

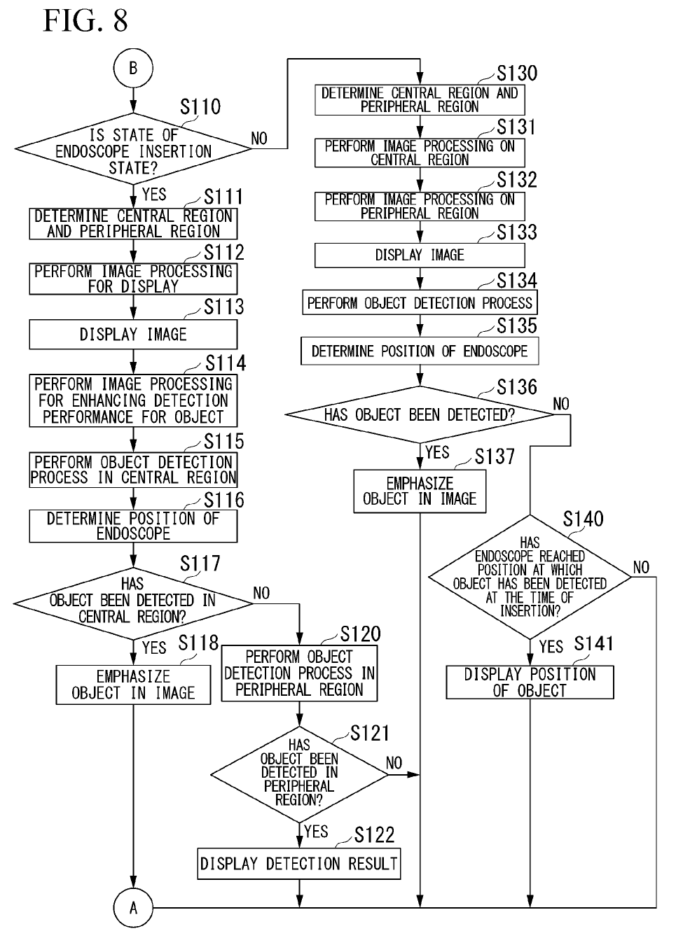

An example of the operation of the image-processing device 20 will be described with reference to FIGS. 7 and 8. FIGS. 7 and 8 show an example of a procedure of a process executed by the image-processing device 20.

### Step S100

A user inputs various examination conditions to the image-processing device 20. The examination information generation unit 21 determines whether an examination condition has been input. When an examination condition has not been input, the examination information generation unit 21 repeatedly executes this determination. When an examination condition has been input, the examination information generation unit 21 generates examination information. Thereafter, Step S101 is executed.

### Step S101

After the examination condition has been input, the processing control unit 24 causes the imaging unit 130 to start imaging.

### Step S102

The state determination unit 254 determines whether the state of the endoscope 13 is either an insertion state, an extraction state, or an examination state using the aforementioned method. The state determination unit 254 may determine the state of the endoscope 13 using a partial area of an image generated by the imaging unit 130 without using the entire image.

### Step S103

The endoscope 13 may rotate around a center axis thereof with movement of the endoscope 13. The state determination unit 254 analyzes a plurality of images generated by the imaging unit 130 and determines a rotation state of the endoscope 13. The rotation state includes a rotation direction and the amount of rotation. When an acceleration sensor is provided at the distal end of the endoscope 13, the state determination unit 254 may determine the rotation state of the endoscope 13 based on a signal output from the acceleration sensor. Rotation state information indicating the rotation state is recorded on the recording medium 30.

### Step S104

The processing control unit 24 determines whether the state of the endoscope 13 is the insertion state or the extraction state based on the process result in Step S102. When the state of the endoscope 13 is the insertion state or the extraction state, Step S110 is executed. When the state of the endoscope 13 is a state other than the insertion state and the extraction state, Step S150 is executed.

### Step S110

The processing control unit 24 determines whether the state of the endoscope 13 is the insertion state based on the process result in Step S102. When the state of the endoscope 13 is the insertion state, Step S111 is executed. When the state of the endoscope 13 is the extraction state, Step S130 is executed.

### Step S111

The central region determination unit 22 determines a central region in the image based on the boundary information 32 recorded on the recording medium 30. The central region determination unit 22 outputs information indicating the position of the central region to the image-processing unit 25. The peripheral region determination unit 23 determines a peripheral region in the image based on the boundary information 32 recorded on the recording medium 30. The peripheral region determination unit 23 outputs information indicating the position of the peripheral region to the image-processing unit 25.

### Step S112

The peripheral image-processing unit 251 executes peripheral image processing for display on the image of the peripheral region determined by the peripheral region determination unit 23. As described above, the peripheral image-processing unit 251 deletes the image of the peripheral region from the image generated by the imaging unit 130. Alternatively, the peripheral image-processing unit 251 executes a thinning-out process on the image generated by the imaging unit 130 and thins out pixels of the image of the peripheral region. In the following description, it is assumed that the peripheral image-processing unit 251 deletes the image of the peripheral region. The image correction unit 255 corrects the image processed by the peripheral image-processing unit 251. The image processed by the image correction unit 255 is output to the display control unit 26.

The central image-processing unit 250 may execute central image processing for display on the image of the central region determined by the central region determination unit 22. For example, the central image-processing unit 250 may execute processing for improving the image quality of the image of the central region.

### Step S113

The display control unit 26 outputs the image processed in Step S112 to the display unit 40 and causes the display unit 40 to display the image.

### Step S114

The central image-processing unit 250 and the peripheral image-processing unit 251 execute image processing for improving the detection performance for an object and generates an image for object detection. For example, the central image-processing unit 250 executes processing for improving the image quality on the image of the central region, and the peripheral image-processing unit 251 executes the processing for improving the image quality on the image of the peripheral region. At this time, the intensity of the processing executed on the image of the peripheral region is higher than or equal to the intensity of the processing executed on the image of the central region. The central image-processing unit 250 may not execute the image processing, but only the peripheral image-processing unit 251 may execute the image processing.

### Step S115

The central object detection unit 252 detects an object from the image of the central region in the image processed in Step S114. When the object has been detected, first position information indicating the position of the object in the image of the central region is recorded on the recording medium 30.

### Step S116

The position determination unit 256 determines a current position of the distal end of the endoscope 13 using the aforementioned method and generates second position information indicating the position. The second position information is recorded on the recording medium 30. The first position information recorded in Step S115 and the second position information recorded in Step S116 are assiciated with each other. In addition, state information indicating the insertion state is assiciated with the first position information and the second position information.

### Step S117

The processing control unit 24 determines whether an object has been detected in the image of the central region based on the processing result in Step S115. When the object has been detected in the image of the central region, Step S118 is executed. When the object has not been detected in the image of the central region, Step S120 is executed.

### Step S118

The display control unit 26 emphasizes the object in the central region in the image displayed on the display unit 40. For example, the display control unit 26 displays a frame around the object. After Step S118 has been executed, Step S102 is executed.

### Step S120

The peripheral object detection unit 253 detects an object from the image of the peripheral region in the image processed in Step S114. When the object has been detected, first position information indicating the position of the object in the image of the peripheral region is recorded on the recording medium 30. The first position information recorded in Step S115 and the second position information recorded in Step S120 are associated with each other. In addition, state information indicating the insertion state is associated with the first position information and the second position information.

### Step S121

The processing control unit 24 determines whether an object has been detected in the image of the peripheral region based on the processing result in Step S120. When the object has been detected in the image of the peripheral region, Step S122 is executed. When the object has not been detected in the image of the peripheral region, Step S102 is executed.

### Step S122

The display control unit 26 displays a detection result indicating that the object has been detected in the image of the peripheral region on the image displayed on the display unit 40. The display control unit 26 may display information indicating the position of the object in the peripheral region on the image displayed on the display unit 40. For example, the display control unit 26 may display an arrow indicating the position indicated by the first position information recorded on the recording medium 30.

At this time, the display control unit 26 may display information indicating the type of the object in addition to the information indicating the position of the object on the image displayed on the display unit 40. The information indicating the type of the object may include information indicating features of the object. After Step S122 has been executed, Step S102 is executed.

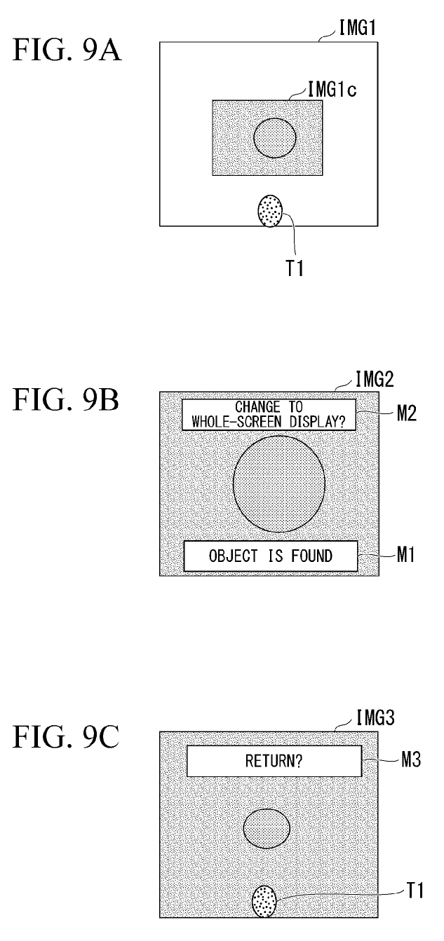

FIG. 9A shows an image IMG1 generated by the imaging unit 130. The image IMG1 corresponds to the entire imaging field of view of the imaging unit 130. An object T1 appears in the peripheral region of the image IMG1. Since the peripheral image-processing unit 251 deletes the image of the peripheral region from the image IMG1 in Step S112, only the image IMG1c of the central region is displayed on the display unit 40.

FIG. 9B shows an image IMG2 displayed on the display unit 40. The image IMG1c is enlarged and displayed as the image IMG2 on the display unit 40. The display control unit 26 displays a message M1 indicating that the object T1 has been detected on the image IMG2. Since the object T1 does not appear in the image IMG1c, the user cannot see the object T1. However, since the message M1 is displayed, the user can ascertain that the object T1 has been detected.

When the imaging field of view of the imaging unit 130 of the endoscope 13 is divided into a central region including the center of the imaging field of view and a peripheral region other than the central region, the central image-processing unit 250 may execute image processing for display on the image of the central region generated by the imaging unit 130. At this time, the central image-processing unit 250 may generate an image with which a user operating the endoscope 13 is likely to concentrate on an insertion operation.

A predetermined lesioned part or the like may appear in the peripheral region of the image generated by the imaging unit 130. In order to effectively use such information, the peripheral object detection unit 253 detects an object from the image of the peripheral region. The display control unit 26 displays a detection result of the object on the display unit 40 that displays the image of the central region. Accordingly, the user can reliably recognize information to be acquired from the regions.

Particularly, when the user is performing the insertion operation while determining an inward direction and a depth direction of a bent thin lumen, the user pays attention to a method of advancing rather than the lesioned part in the peripheral region of the image. At this timing, the user is likely to overlook image information serving as other clues. Here, for example, since an AI function or the like monitors the peripheral region, the user can concentrate on the operation with ease.

The image of the central region has a rectangular shape. The detection result of the object is an icon including information indicating a direction directed from the center of the rectangular shape to a side closest to the position at which the object has been detected out of four sides of the rectangular shape. Accordingly, the user can ascertain in what direction to move the distal end of the endoscope 13. The display control unit 26 may display a diagram showing a lever for bending the distal end of the endoscope 13, an operation direction of the lever, and a direction in which the distal end of the endoscope 13 is bent on the display unit 40.

When an object is a biological body, the object is highly likely to be deformed. Since the user performs a remote operation using the lever to bend the distal end of the endoscope 13, the direction in which the distal end of the endoscope 13 is bent is likely to have an error. Accordingly, the user is likely to miss the object unless bending the distal end of the endoscope 13 with a clear instruction without any correction. As described above, the display unit 40 may display information indicating whether there is an object. In addition, when an inference model for discriminating the type of an object is mounted, the display unit 40 may display incidental information thereof. The display unit 40 may display features such as the size or the color of the object.

The display control unit 26 displays a message M2 on the image IMG2 for encouraging the user to display the entire image. When the user performs a predetermined operation, the display control unit 26 displays an image IMG3 shown in FIG. 9C on the display unit 40. The image IMG3 corresponds to the entire imaging field of view of the imaging unit 130. The object T1 appears in the peripheral region of the image IMG3. The user can ascertain the object T1.

When the state of the endoscope 13 is the insertion state, Step S112 or the like is repeatedly executed. In a case where the peripheral object detection unit 253 does not detect an object in the image of the peripheral region, the peripheral image-processing unit 251 deletes the image of the peripheral region from the entire image of the imaging field of view of the imaging unit 130 in Step S112. After the peripheral object detection unit 253 has detected an object in the image of the peripheral region, the peripheral image-processing unit 251 does not delete the image of the peripheral region from the entire image of the imaging field of view of the imaging unit 130 in Step S112. Accordingly, the entire image IMG3 generated by the imaging unit 130 is displayed on the display unit 40.

The display control unit 26 displays a message M3 on the image IMG3 for encouraging the user to return to a state in which an image of the same range as the image IMG2 is displayed. When the user performs a predetermined operation, the display control unit 26 displays the same image as the image IMG2 on the display unit 40. When an object has been detected in the image of the peripheral region, the display control unit 26 may not display the image IMG2 but display the image IMG3 on the display unit 40.

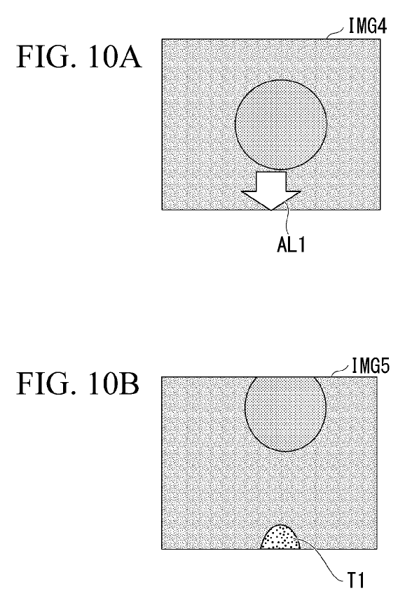

The display control unit 26 may display an image IMG4 shown in FIG. 10A on the display unit 40 instead of the image IMG2. The display control unit 26 may display an alarm AL1 on the image IMG4. The alarm AL1 indicates a position indicated by the first position information recorded on the recording medium 30. That is, the alarm AL1 indicates the position of the object. The alarm AL1 is an arrow and indicates that the object is present downward in the image IMG4.

After the image IMG4 has been displayed, the user may turn the distal end of the endoscope 13 to the object. In this case, the display control unit 26 displays, for example, an image IMG5 shown in FIG. 10B on the display unit 40. Part of the object T1 appears in the image IMG5.

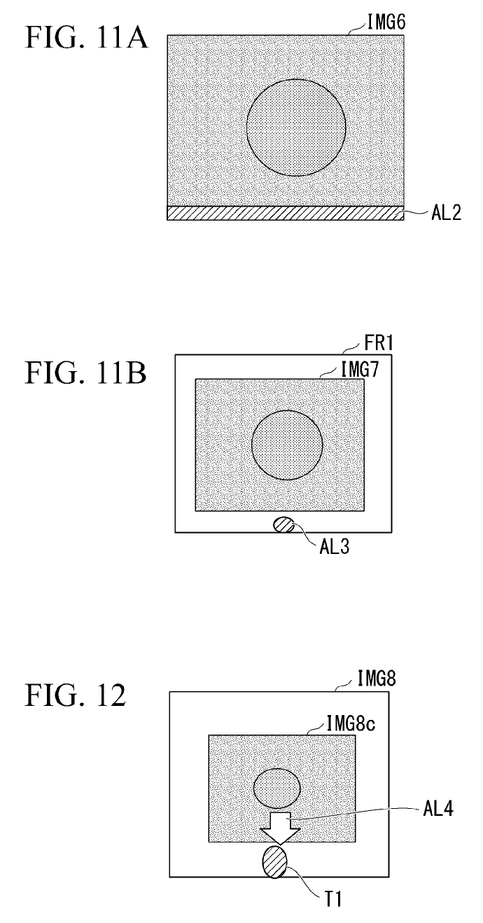

The display control unit 26 may display an image IMG6 shown in FIG. 11A on the display unit 40 instead of the image IMG2. The display control unit 26 may display an alarm AL2 on the image IMG6. The position of the alarm AL2 indicates the position indicated by the first position information recorded on the recording medium 30. That is, the position of the alarm AL2 indicates the position of the object. Since the alarm AL2 is displayed in a lower part of the image IMG6, the alarm AL2 indicates that the object is present downward in the image IMG6.

The display control unit 26 may display an image IMG7 shown in FIG. 11B on the display unit 40 instead of the image IMG2. The image IMG7 is generated by deleting the image of the peripheral region from the image generated by the imaging unit 130. The display control unit 26 may display a frame FR1 indicating a range of the image generated by the imaging unit 130 on the display unit 40. The display control unit 26 may display an alarm AL3 outside the image IMG7 and inside of the frame FR1. The position of the alarm AL3 indicates the position indicated by the first position information recorded on the recording medium 30. That is, the position of the alarm AL3 indicates the position of the object. Since the alarm AL3 is displayed in a lower part of the image IMG7, the alarm AL3 indicates that the object is present downward in the image IMG7.

### Step S130

The central region determination unit 22 determines a central region of the image based on the boundary information 32 recorded on the recording medium 30. The central region determination unit 22 outputs information indicating the position of the central region to the image-processing unit 25. The peripheral region determination unit 23 determines a peripheral region of the image based on the boundary information 32 recorded on the recording medium 30. The peripheral region determination unit 23 outputs information indicating the position of the peripheral region to the image-processing unit 25. The boundary between the central region and the peripheral region in Step S130 may be different from the boundary between the central region and the peripheral region in Step S111. For example, since the image of the central region is significant in the insertion state, the central region in Step S111 may be larger than the central region in Step S130.

### Step S131

The central image-processing unit 250 executes image processing on the image of the central region determined by the central region determination unit 22.

### Step S132

The peripheral image-processing unit 251 executes image processing on the image of the peripheral region determined by the peripheral region determination unit 23. The image correction unit 255 corrects the images processed by the central image-processing unit 250 and the peripheral image-processing unit 251. The images processed by the image correction unit 255 are output to the display control unit 26.

The image processing in Step S132 may be the same as the image processing in Step S131. For example, the central image-processing unit 250 and the peripheral image-processing unit 251 may execute processing for improving the visibility. The processing for improving the visibility may be the same as the processing for improving the image quality. The intensity of processing executed on the image of the peripheral region may be the same as the intensity of processing executed on the image of the central region. Accordingly, the visibility of the image of the peripheral region processed by the peripheral image-processing unit 251 may be the same as the visibility of the image of the peripheral region processed by the central image-processing unit 250.

### Step S133

The display control unit 26 outputs the images processed in Steps S131 and S132 to the display unit 40 and causes the display unit 40 to display the images.

### Step S134

The central object detection unit 252 detects an object in the image of the central region processed in Step S131. When the object has been detected, first position information indicating the position of the object in the image of the central region is recorded on the recording medium 30. The peripheral object detection unit 253 detects an object in the image of the peripheral region processed in Step S132. When the object has been detected, first position information indicating the position of the object in the image of the peripheral region is recorded on the recording medium 30.

### Step S135

The position determination unit 256 determines a current position of the distal end of the endoscope 13 and generates second position information indicating the position. The second position information is recorded on the recording medium 30. The first position information recorded in Step S134 and the second position information recorded in Step S135 are associated with each other. In addition, state information indicating the extraction state is associated with the first position information and the second position information.

### Step S136

The processing control unit 24 determines whether an object has been detected in at least one of the image of the central region and the image of the peripheral region based on the processing result in Step S134. When the object has been detected in at least one of the image of the central region and the image of the peripheral region, Step S137 is executed. When the object has not been detected in any of the image of the central region and the image of the peripheral region, Step S140 is executed.

### Step S137

The display control unit 26 emphasizes the object in the image displayed on the display unit 40. For example, the display control unit 26 displays a frame at the position indicated by the first position information recorded on the recording medium 30. Accordingly, a frame is displayed around the object. After Step S137 has been executed, Step S102 is executed.

### Step S140

The processing control unit 24 determines whether the second position information indicating the same position as the position determined in Step S135 is recorded on the recording medium 30. Accordingly, the processing control unit 24 determines whether the endoscope 13 has reached the position at which the object has been detected in the insertion state. When the object detected in the insertion state has been detected again in the extraction state, the second position information indicating the same position as the position determined in Step S135 is recorded on the recording medium 30. In this case, the processing control unit 24 determines that the endoscope 13 has reached the position at which the object has been detected in the insertion state. When the second position information indicating the same position as the position determined in Step S135 is not recorded on the recording medium 30, the processing control unit 24 determines that the endoscope 13 has not reached the position at which the object has been detected in the insertion state. When the endoscope 13 has reached the position at which the object has been detected in the insertion state, Step S141 is executed. When the endoscope 13 has not reached the position at which the object has been detected in the insertion state, Step S102 is executed.

### Step S141

The display control unit 26 acquires the first position information associated with the second position information indicating the same position as the position determined in Step S135 from the recording medium 30. The display control unit 26 displays the position indicated by the first position information on the image displayed on the display unit 40. The rotation state of the endoscope 13 in the insertion state and the rotation state of the endoscope 13 in the extraction state may be different. The display control unit 26 may correct the position indicated by the first position information in consideration of the change in the rotation state of the endoscope 13.

### Step S150

The processing control unit 24 determines whether the first position information that is associated with the state information indicating the insertion state and indicates the position in the image of the peripheral region is recorded on the recording medium 30. When the peripheral object detection unit 253 has detected an object in the insertion state, the first position information that is associated with the state information indicating the insertion state and indicates the position in the image of the peripheral region is recorded on the recording medium 30 in Step S120. When the first position information that is associated with the state information indicating the insertion state and indicates the position in the image of the peripheral region is recorded on the recording medium 30, the processing control unit 24 determines whether the distal end of the endoscope 13 is directed to the position indicated by the first position information.

When the distal end of the endoscope 13 is directed to the position indicated by the first position information, the state of the endoscope 13 is the examination state and Step S151 is executed. When the distal end of the endoscope 13 is not directed to the position indicated by the first position information, Step S160 is executed. In order to avoid complication of explanation, it is assumed below that the state of the endoscope 13 is maintained in the examination state while the user is inserting the endoscope 13 into an examination target. When the first position information that is associated with the state information indicating the insertion state and indicates the position in the image of the peripheral region is not recorded on the recording medium 30, Step S160 is executed.

When Step S122 is executed, the detection result indicating that an object has been detected in the image of the peripheral region is displayed on the image. At this time, since the image of the peripheral region is not displayed, the object is not displayed. The user determines that the object has been detected and turns the distal end of the endoscope 13 to the object in order to observe the object. In the image generated by the imaging unit 130, the object approaches the center of the imaging field of view. In Step S150, this state is determined.

### Step S151

The central region determination unit 22 outputs information indicating the position of the central region wider than the central region determined in Step S111 to the image-processing unit 25. The peripheral region determination unit 23 outputs information indicating the position of the peripheral region narrower than the peripheral region determined in Step S111 to the image-processing unit 25. When an object approaches the center of the imaging field of view in a plurality of images consecutively generated by the imaging unit 130, the central region in the plurality of images is gradually enlarged and the peripheral region in the plurality of images is gradually reduced.

### Step S152

The peripheral image-processing unit 251 deletes the image of the peripheral region determined by the peripheral region determination unit 23. The peripheral region deleted in Step S152 is smaller than the peripheral region deleted in Step S112. The image correction unit 255 corrects the image processed by the peripheral image-processing unit 251. The image processed by the image correction unit 255 is output to the display control unit 26.

### Step S153

The display control unit 26 outputs the image processed in Step S152 to the display unit 40 and causes the display unit 40 to display the image.

### Step S154

The display control unit 26 displays information indicating the position of the object in the peripheral region on the image displayed on the display unit 40.

FIG. 12 shows an image IMG8 generated by the imaging unit 130. The image IMG8 corresponds to the entire imaging field of view of the imaging unit 130. An object T1 appears in the peripheral region of the image IMG8. Since the peripheral image-processing unit 251 deletes the image of the peripheral region in Step S152, only the image IMG8c of the central region is displayed on the display unit 40. The display control unit 26 displays an alarm AL4 on the image IMG8c. The size of the image IMG8 is the same as the size of the image IMG1 shown in FIG. 9A. The image IMG8c is larger than the image IMG1c shown in FIG. 9A.

### Step S160

The central image-processing unit 250 or the peripheral image-processing unit 251 executes image processing for display on the entire image generated by the imaging unit 130.

### Step S161

The display control unit 26 outputs the image processed in Step S160 to the display unit 40 and causes the display unit 40 to display the image.

### Step S162

The central object detection unit 252 or the peripheral object detection unit 253 detects an object from the entire image processed in Step S160.

### Step S163

When the object has been detected in Step S162, the display control unit 26 displays the detection result indicating that the object has been detected on the image displayed on the display unit 40. For example, the display control unit 26 displays a frame around the object. After Step S163 has been executed, Step S102 is executed.

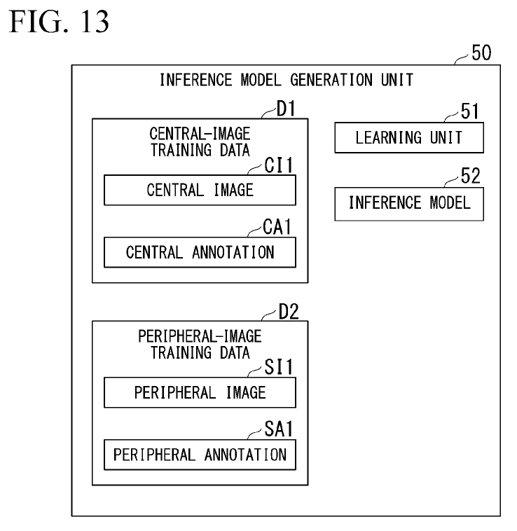

The central object detection unit 252 and the peripheral object detection unit 253 may detect an object using an inference model (AI) which has been obtained through deep learning using feature information appearing in an image as training data, or the like. The endoscope system 1 may include an inference model generation unit 50 shown in FIG. 13. FIG. 13 shows the configuration of the inference model generation unit 50. The inference model generation unit 50 may be included in the image-processing device 20.

The inference model generation unit 50 includes a learning unit 51. The learning unit 51 may generate training data by adding an annotation to a frame (a frame of interest) in which a predetermined image feature (such as a lesioned part) occurs out of video data including a series of frames acquired from start to end of an examination. The learning unit 51 may generate an inference model 52 by executing learning such that a frame of interest is output when consecutive frames of a video acquired by the endoscope 13 are input.

For example, the learning unit 51 generates central-image training data D1 and peripheral-image training data D2. The central-image training data D1 includes a central image CI1 and a central annotation CA1. The central image CI1 is an image of a central region of the image generated by the imaging unit 130. The central annotation CA1 is added to the central image CI1. The peripheral-image training data D2 includes a peripheral image SI1 and a peripheral annotation SA1. The peripheral image SI1 is an image of a peripheral region of the image generated by the imaging unit 130. The peripheral annotation SA1 is added to the peripheral image SI1.

The learning unit 51 generates an inference model 52 by executing machine learning using images generated by the imaging unit 130 in a plurality of cases and annotations that are results of determination for a lesioned part in the images as training data. The inference model 52 is, for example, a neural network and is generated through deep learning. The inference model 52 is not limited to a neural network and may be another machine learning model that can output information in response to an input image. The inference model 52 is recorded on the recording medium 30.

For example, the central-image training data D1 (first training data) used in the machine learning includes both a first learning image that has been generated by the imaging unit 130 and on which the first image processing has been executed by the central image-processing unit 250 and a central annotation CA1 (a first annotation) indicating an object in a central region (a central image CI1) of the first learning image. In addition, the peripheral-image training data D2 (second training data) used in the machine learning includes both a second learning image that has been generated by the imaging unit 130 and on which the second image processing has been executed by the peripheral image-processing unit 251 and a peripheral annotation SA1 (a second annotation) indicating an object in a peripheral region (a peripheral image SI1) of the second learning image. The processing control unit 24 (an acquisition unit) acquires the inference model obtained through the machine learning from the recording medium 30. The central object detection unit 252 or the peripheral object detection unit 253 detects an object in the image generated by the imaging unit 130 using the inference model acquired by the processing control unit 24.

The first learning image on which the image processing for display (Step S112) has been executed by the central image-processing unit 250 is used to generate the inference model used in Step S115. In addition, the second learning image on which the image processing for display (Step S112) has been executed by the peripheral image-processing unit 251 is used to generate the inference model used in Step S120.

The first learning image on which the image processing (Step S131) has been executed by the central image-processing unit 250 and the second learning image on which the image processing for display (Step S132) has been executed by the peripheral image-processing unit 251 are used to generate the inference model used in Step S134.

The learning unit 51 may generate a first inference model through first machine learning using a learning image generated by the imaging unit 130 and a central annotation CA1 (a first annotation) indicating an object in the central region (the central image CI1) of the learning image as the central-image training data D1 (first training data). The learning unit 51 may generate a second inference model through second machine learning using the learning image and a peripheral annotation SA1 (a second annotation) indicating an object in the peripheral region (the peripheral image SI1) of the learning image as the peripheral-image training data D2 (second training data). The processing control unit 24 (the acquisition unit) may acquire the first inference model and the second inference model from the recording medium 30. The central object detection unit 252 may detect an object from the image of the central region in the image generated by the imaging unit 130 using the first inference model acquired by the processing control unit 24. The peripheral object detection unit 253 may detect an object from the image of the peripheral region in the image generated by the imaging unit 130 using the second inference model acquired by the processing control unit 24.

“Deep learning” contains processes of “machine learning” using a neural network, and the processes are structured in multiple layers. A representative example is a “forward-propagation neural network” of executing determination while sending information forwardly. In the most simple example, the neural network has only to include three layers that are an input layer including N1 neurons, an intermediate layer including N2 neurons given as parameters, and an output layer including N3 neurons corresponding to the number of classes to be determined. By coupling the neurons in the input layer and the intermediate layer by coupling weights, coupling the neurons in the intermediate layer and the output layer by coupling weights, and adding bias values to the intermediate layer and the output layer, logic gates can be easily formed. Three layers may be used for the purpose of simple determination, and a method of combining a plurality of features may be learned in the course of machine learning by increasing the number of intermediate layers. In recent years, 9 to 152 intermediate layers can be practically used in view of a time required for learning, determination accuracy, and energy consumption. A “convolutional neural network” using minimum processes accompanying a process called “convolution” of compressing features of an image may be used. The convolutional neural network is strong in recognition of motion and patterns. Alternatively, a “recurrent neural network” (an all-coupling recurrent neural network) that can handle more complex information may be used. In the recurrent neural network, information propagates bidirectionally to correspond to information analysis in which meanings change according to the sequence. In addition, a technique such as support vector machine or support vector recurrence is used as a pattern recognition model using supervised learning. Weights, filter coefficients, and offsets of discriminators are calculated in such learning. In addition, there is also a technique using a logistic regression process.

A general-purpose arithmetic processing circuit such as a CPU or a field-programmable gate array (FPGA) may be used to execute learning, and a circuit such as a graphic processing unit (GPU) or a tensor processing unit (TPU) characterized in matrix calculation may be used since most processes in the neural network are multiplication of matrices. In recent years, a “neural network processing unit (NPU)” that is hardware specific to artificial intelligence (AI) may be designed to be integrated with a circuit such as a CPU and serve as a part of a processing circuit.

For example, when an inference model generated through learning using only images acquired by general imaging and having the horizontal and vertical directions aligned is used, there is a likelihood that correct inference may not be performed on images having vertical or horizontal differences. Accordingly, it is possible to perform correct determination by reading the above-described information from inference information and executing inference on images acquired in consideration of horizontal or vertical information from a posture sensor. An idea of adding horizontal and vertical information and determining images before executing inference using the inference model is effective. It is preferable that the endoscope system 1 store information of such conditions and have a sensor for correcting an image. Specifications and performance of an inference engine change according to whether such constraints are added at the time of learning. Accordingly, this trial and error may be performed in parallel with an annotation operation, or the trial and error may be displayed.

Similarly, when learning using only images captured at a position separated by a specific distance from an object is executed, correct inference cannot be executed on an image captured at a position separated by a distance other than the specific distance. In inference using an inference model generated based on such images, it is possible to improve accuracy based on an idea of enlarging an image of a far object and artificially using an image of a nearby object in order to cancel the difference in distance. In this case, a distance sensor or the like is used together, and correction for complementing the difference between a state of actual enlargement or reduction of an image and a state of training data is executed at the time of inference of the image. The image-processing device 20 may include a memory for storing information indicating the training data used to generate the inference model and correct an image such that the inference model can correctly execute inference using the above-described information when the inference using the inference model is executed. A user may be aware whether such correction is necessary in the annotation operation. As in the present embodiment, the idea of enabling verification of provisional learning in the annotation operation is significant.

It is difficult for a compact inference engine mounted in an information terminal product such as a camera or a portable device to execute learning for highly accurate determination with smaller layers. Since learning requires a time, there is need for an idea associated with a method of executing accurate annotation and learning. When an inference model is generated, specifications of the inference model are changed according to images used for learning, and thus efficient learning may be executed in cooperation with information at the time of learning. Therefore, information indicating what learning has been executed may be set in the annotation operation, and this information may be recorded as part of inference information in a recording unit of an information acquisition device.

As described above, in “supervised learning,” a “relationship between an input and an output” is learned using training data of which the output is determined by an annotation, and inference with high reliability under specific conditions is executed. On the other hand, the image-processing device 20 may acquire an inference model that can cope with more complex situations using a technique of “unsupervised learning” of learning a “data structure.”

The image-processing device 20 may use a technique of learning an “action for maximizing values and effects” called “reinforcement learning.” In this technique, learning is executed such that a rule for enhancing state/action values is searched for. Trial and error are made until values of a next state other than a current state are estimated and enhanced or specific rewards are acquired, and the results of the trial and error are reflected in learning. Training data may be used to verify results of learning. In this technique, the output of an answer acquired by the annotation is not learned as it is, but learning is executed such that a more correct answer can be obtained. Accordingly, it is possible to cope with an unknown situation.

This inference may be used together with supervised learning, or the inference may be executed using supervised learning after the inference has been executed using unsupervised learning. Annotation data can also be used as verification data for such “unsupervised learning” and “reinforcement learning.”

When a machine is made to determine something, a human being needs to teach the machine a determination method. Here, a technique of executing determination of an image through machine learning has been employed, and a rule-based technique of causing a human being to apply an experimental rule or a rule acquired in heuristics to determination may be used.

As described above, the image-processing device 20 can execute image processing which is appropriate for the roles of the central region and the peripheral region of an image.

While preferred embodiments of the invention have been described and shown above, it should be understood that these are examples of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An image-processing device comprising a processor configured to:
determine whether a state of an endoscope is either a first state in which the endoscope is advancing inward in an examination target or a second state other than the first stat;
when an imaging field of view of an image sensor of the endoscope is divided into a first region including a center of the imaging field of view and a second region other than the first region and the state of the endoscope is the first state, execute image processing of setting a visibility of an image of the second region to be lower than a visibility of an image of the first region to generate an image for display; and
execute image processing of setting a detection performance for an object in the image of the second region to be higher than or equal to a detection performance for an object in the image of the first region to generate an image for object detection when the state of the endoscope is the first state.

2. The image-processing device according to claim 1, wherein the processor is configured not to execute the image processing of setting the visibility of the image of the second region to be lower than the visibility of the image of the first region when the state of the endoscope is the second state.

3. The image-processing device according to claim 1, wherein the processor is configured to:
detect an object from the image of the second region in the image for object detection when the state of the endoscope is the first state; and
display a detection result indicating that the object has been detected along with the image of the first region on which the image processing has been executed on a display when the state of the endoscope is the first state.

4. The image-processing device according to claim 3, wherein the processor is configured to display position information indicating a position of the object in the image of the second region as the detection result on the display.

5. The image-processing device according to claim 1, wherein the processor is configured to:
detect an object from the image of the second region in the image for object detection and store first position information indicating a position of the object in the image of the second region and second position information indicating a position of the object in the examination target on a recording medium when the state of the endoscope is the first state; and
display the first position information along with an image generated by the image sensor on a display when the state of the endoscope is the second state and a position of the endoscope matches the position indicated by the second position information.

6. The image-processing device according to claim 1, wherein the processor is configured to delete the image of the second region when the state of the endoscope is the first state.

7. The image-processing device according to claim 6,
wherein the processor is configured to detect an object from the image of the second region in the image for object detection when the state of the endoscope is the first state,
wherein the processor is configured to delete the image of the second region when the object has not been detected, and
wherein the processor is configured not to delete the image of the second region when the object has been detected.

8. The image-processing device according to claim 6,
wherein the processor is configured to detect an object from the image of the second region in the image for object detection when the state of the endoscope is the first state, and
wherein the first region in a plurality of images consecutively generated by the image sensor is gradually enlarged and the second region in the plurality of images is gradually reduced when the object in the plurality of images approaches the center of the imaging field of view.

9. The image-processing device according to claim 1, wherein the processor is configured to execute a thinning-out process on the image of the second region when the state of the endoscope is the first state.

10. The image-processing device according to claim 1, wherein the processor is configured to correct an image generated by the image sensor based on optical characteristics of the endoscope.

11. An image-processing device comprising a processor configured to:
when an imaging field of view of an image sensor of an endoscope is divided into a first region including a center of an imaging field of view and a second region other than the first region, execute first image processing on an image of the first region generated by the image sensor;
execute second image processing on an image of the second region generated by the image sensor;
detect an object in the image on which the second image processing has been executed; and
display a detection result of the object along with the image of the first region on which the first image processing has been executed on a display.

12. The image-processing device according to claim 11, wherein a boundary between the first region and the second region is switchable between that in a first state in which the endoscope is inserted into an examination target and is caused to advance inwardly in the examination target and that in a second state in which the endoscope is extracted from the examination target.

13. An image-processing device comprising a processor configured to:
when an imaging field of view of an image sensor of an endoscope is divided into a first region including a center of the imaging field of view and a second region other than the first region, acquire an inference model, which is obtained through machine learning using a first learning image and a first annotation as first training data and using a second learning image and a second annotation as second training data, from a recording medium,
wherein first image processing has been executed on the first learning image, the first annotation indicates an object in the first region of the first learning image, second image processing has been executed on the second learning image, and the second annotation indicates an object in the second region of the second learning image; and
detect an object in an image generated by the image sensor using the inference model.

14. An image-processing device comprising a processor configured to:
when an imaging field of view of an image sensor of an endoscope is divided into a first region including a center of the imaging field of view and a second region other than the first region, acquire a first inference model, which is obtained through first machine learning using a learning image generated by an image sensor of an endoscope and a first annotation indicating an object in the first region of the learning image as first training data, from a recording medium;
acquire a second inference model, which is obtained through second machine learning using the learning image and a second annotation indicating an object in the second region of the learning image as second training data, from the recording medium; and
detect an object in an image generated by the image sensor using the first inference model and the second inference model.

15. An image-processing method comprising:
determining whether a state of an endoscope is either a first state in which the endoscope is advancing inward in an examination target or a second state other than the first state;
when an imaging field of view of an image sensor of the endoscope is divided into a first region including a center of the imaging field of view and a second region other than the first region and the state of the endoscope is the first state, executing image processing of setting a visibility of an image of the second region to be lower than a visibility of an image of the first region to generate an image for display; and
executing image processing of setting a detection performance for an object in the image of the second region to be higher than or equal to a detection performance for an object in the image of the first region to generate an image for object detection when the state of the endoscope is the first state.

16. An image-processing method comprising:
when an imaging field of view of an image sensor of an endoscope is divided into a first region including a center of the imaging field of view and a second region other than the first region, executing first image processing on an image of the first region generated by the image sensor;
executing second image processing on an image of the second region generated by the image sensor;
detecting an object in the image on which the second image processing has been executed; and
displaying a detection result of the object along with the image of the first region on which the first image processing has been executed on a display.

17. An image-processing method comprising:
when an imaging field of view of an image sensor of an endoscope is divided into a central region including a center of the imaging field of view and a peripheral region other than the central region, executing image processing for display on an image of the central region generated by the image sensor;
detecting an object in an image of the peripheral region generated by the image sensor; and
displaying a detection result of the object on a display on which the image of the central region is displayed.

18. The image-processing method according to claim 17,
wherein the image of the central region has a rectangular shape, and
wherein the detection result of the object is an icon including at least one of information indicating presence of the object, information indicating a type of the object, and information indicating a direction directed from a center of the rectangular shape to a side closest to a position at which the object has been detected out of four sides of the rectangular shape.

19. A non-transitory computer-readable recording medium storing a program causing a computer to execute:
determining whether a state of an endoscope is either a first state in which the endoscope is advancing inward in an examination target or a second state other than the first state;
when an imaging field of view of an image sensor of the endoscope is divided into a first region including a center of the imaging field of view and a second region other than the first region and the state of the endoscope is the first state, executing image processing of setting a visibility of an image of the second region to be lower than a visibility of an image of the first region to generate an image for display; and
executing image processing of setting a detection performance for an object in the image of the second region to be higher than or equal to a detection performance for an object in the image of the first region to generate an image for object detection when the state of the endoscope is the first state.

20. A non-transitory computer-readable recording medium storing a program causing a computer to execute:
when an imaging field of view of an image sensor of an endoscope is divided into a first region including a center of the imaging field of view and a second region other than the first region, executing first image processing on an image of the first region generated by the image sensor;
executing second image processing on an image of the second region generated by the image sensor;
detecting an object in the image on which the second image processing has been executed; and
displaying a detection result of the object along with the image of the first region on which the first image processing has been executed on a display.
